(19) United States
(12) Patent Application Publication
(10) Pub. No.: US 2010/0094151 A1
(43) Pub. Date: Apr. 15, 2010

(54) Stethoscope Timer Attachment and Clip Holder Therefor

(76) Inventors: Jeff Baker, Orlando, FL (US); Seth Freytag, Windermere, FL (US); Mark Bunker, Orlando, FL (US)

Correspondence Address: Timothy H. Van Dyke;Beusse Wolter Sanks Mora & Maire, P.A., Suite 2500, 390 North Orange Avenue, Orlando, FL, 32801, US

(21) Appl. No.: 12/248,073

(22) Filed: Oct. 9, 2008

(51) Int. Cl. A61B 5/02

(52) U.S. Cl. 600/528

(57) ABSTRACT

A stethoscope timer attachment and clip holder therefore comprising a timing device affixed to a tube of a stethoscope, and a holster to receive the timing device in an inverted position, wherein said holster is connected to a waist band of a pair of pants of a health care professional to carry the stethoscope thereon.

### FIELD OF THE INVENTION

The present invention relates to stethoscope accessories, and more particularly, a stethoscope timer attachment and clip holder therefore.

### BACKGROUND

Measurement of heart rate is relatively common in medicine. Heart or pulse rate measurements are used to assist with determination of the physical health of a patient as well as to determine the extent of any pathologies. One way to measure heart rate is to feel for a pulse, usually done by pressing a finger against a specific location on the wrist or neck of a patient. The specific location preferred for pulse measurements is one where an arterial blood vessel runs close to the surface of the skin and is not overlaid by excessive fat, bone, or muscle. In another method, medical caregivers, including physicians, nurses, EMTs, paramedics, and the like, use a stethoscope to listen to the sounds of the heart or the sounds of blood moving through the arterial vasculature. In either method, the medical caregiver generally counts pulses for a period of time and performs a mathematical computation to determine the number of pulses that occur per minute. A heart rate is generally measured in units of beats per minute.

Health care professionals, such as doctors, nurses, and emergency medical personnel, etc. often carry a stethoscope with them. To carry the stethoscope, medical personnel either carry the stethoscope in the pocket of their lab coat or drape the stethoscope around their necks. In the first instance, the stethoscope is taking up room in the pocket of the lab coat which the health care professional may desire to use for other items, such as pens, pencils, vials, etc. Further, because the coat pocket is used to carry many different types of items, there may be debris in the pocket that is undersirable to get on a stethoscope. When a health care professional drapes the stethoscope around his/her neck, the stethoscope is not very stable, and may fall off when the health care professional bends over or rushes to an emergency.

U.S. Pat. No. D337,952 issued on Aug. 3, 1993, to Wolff teaches an ornamental design for a combined stop watch and clock for a stethoscope.

U.S. Pat. No. 5,692,657 issued on Dec. 2, 1997, to Kilo et al. teaches a stethoscope holder which may be secured to a piece of apparel, such as a pocket of a lab coat or the waist band of a pair of pants. The stethoscope holder includes a base having a front surface and a back surface. A rib which is generally U-shaped in elevation and L-shaped in cross section, extends out from the front surface of the base. The rib defines a generally U-shaped channel which removably receives either the bell or diaphragm section of the chestpiece. A notch is formed at a bottom of the curved section of the rib and is sized to accept the tubing of the stethoscope. A flexible finger extends from the front surface of the base, below the rib and offset from the center of the base, to hold at least one of the ear pieces of the stethoscope. A spring biased clip is hingedly mounted to the back surface of the base, near the top thereof and is operable to selectively and removably secure the holder to a item of apparel.

U.S. Pat. No. 6,065,563 issued on May 23, 2000, to Stowers teaches a stethoscope supporting device that includes a back plate and front portion for securing a stethoscope to the back plate. Connected to the back plate is a clip for securing the same on an article of clothing.

U.S. Pat. No. 6,484,918 issued on Nov. 26, 2002, to Lefebvre teaches a combined stethoscope integral housing and belt adapter for mounting a stethoscope on the belt of a care giver. The integral housing has a back wall and first and second side walls. The upper portion of each side wall contains an ear piece receptacle. A pair of front walls are spaced apart from each other to form a slot and spaced apart from the back wall to form a opening for a stethoscope chest piece. The belt adapter is hingedly attached to a back upper portion of the housing by an axially mounted rod.

U.S. Publication No. 20040/249298 A1 published on Dec. 9, 2004, to Selevan teaches devices and methods for measuring the heart rate of an animal and more specifically, a human. Heart rate measurements are an integral part of many physical examinations. The devices and methods disclosed herein are useful in any field of medicine where a heart rate is measured. The devices utilize a standard medical stethoscope with a removably mounted timer attached thereto. The stethoscope is used by the medical caregiver to listen to cardiac sounds, and more specifically, the heart beats or arterial blood pulses in conjunction with a sphygmomanometer. A countdown timer is attached to the stethoscope between the “Y” and the auscultation head. The countdown timer is actuated by the medical caregiver and the number of cardiac beats are counted until the timer reaches zero. The timer provides an audible warning of timeout and optionally provides a visual indication of timeout. The proximity of the timer to the stethoscope obviates the need to stare at a watch or clock as it counts out the correct amount of seconds. The number of seconds in the countdown sequence is variable and determined from a series of choices provided to the caregiver.

U.S. Pat. No. D551,348 issued on Sept. 18, 2007, to Lee teaches a ornamental design for a stethoscope tag with pulse timer.

It is apparent now that different stethoscope timers and holders are present in the prior art that are adequate for various purposes. However, the aforementioned references do not disclose the elements of embodiments of the present invention and fail to contemplate the unique and advantageous aspects of various embodiments of the invention.

### SUMMARY OF THE INVENTION

Certain embodiments of the present invention provide a stethoscope timer attachment and clip holder therefore that dramatically decreases the time required for a health care professional using a stethoscope to take a pulse rate, while properly carrying the stethoscope and is simple to use.

One embodiment of the present invention provides a stethoscope timer attachment and clip holder therefore comprising a timing device affixed to a tube of a stethoscope, and a holster to receive the timing device in an inverted position, wherein said holster is engageable to the user such as to a waist band of a pair of pants of a health care professional, or belt, to carry the stethoscope thereon.

Another embodiment pertains to a stethoscope carrying system that includes an attachment device that is affixed to a tube of a stethoscope. The attachment device includes a housing with a channel for passage of a stethoscope tube. The system also includes a holster having a body that comprises an outer shell. The outer shell defines a hollow cavity and has a top end with an opening sized to receive and support the attachment device. The outer shell also has a clip associated therewith for engaging said holster to a belt or waist of a user. In a more specific embodiment, the housing has a three-dimensional configuration and the body has a front wall, back wall, and opposing side walls that form the outer shell. The outer shell defines the cavity to be substantially similar in shape with said three-dimensional configuration so as to create a frictional fit between said holster and said attachment device. Typically, the frictional fit is such that the space between the body and the holster is minimized to prevent rattling of the body when in the holster. In a more specific embodiment, the side walls of the body include a notch for receiving and supporting an ear piece of the stethoscope when the attachment device is in the holster.

The novel features which are considered characteristics of certain embodiments of the present invention are set forth in the appended claims. Embodiments of the invention relating to construction and method of operation embodiments, together with additional advantages thereof, will be best understood from the following description of the specific embodiments when read and understood in connection with the accompanying drawings.

### BRIEF DESCRIPTION OF THE DRAWINGS

The figures of the drawings are briefly described as follows:

FIG. 1 is a front perspective view of an embodiment of the present invention holding a stethoscope to a waist band of a pair of pants;

FIG. 2 is a front perspective view of the timing device attached to the tube of the stethoscope;

FIG. 3 is a bottom perspective view taken in the direction of arrow 3 in FIG. 2;

FIG. 4 is a front view taken in the direction of arrow 4 in FIG. 3 of the timing device per se;

FIG. 5 is a top view taken in the direction of arrow 5 in FIG. 4;

FIG. 6 is a side view taken in the direction of arrow 6 in FIG. 4;

FIG. 7 is a front view of the holster per se; and

FIG. 8 is a side view taken in the direction of arrow 8 in FIG. 7.

FIG. 9 is back view of an embodiment of the present invention.

### REFERENCE NUMERALS UTILIZED IN THE DRAWINGS

10 stethoscope timer attachment and clip holder
12 timing device of attachment 10
14 tube of stethoscope 16
16 stethoscope
18 holster of attachment 10
20 waist band of pair of pants 22
22 pair of pants
24 health care professional
26 snap clamp of timing device 12
28 battery operated count down timer unit in timing device 12
30 housing of timing device 12
32 digital display screen in housing 30
34 timer button on housing 30
36 exam light on bottom 38 of housing 30
38 bottom of housing 30
40 button switch on housing 30
42 light emitting diode for exam light 36
44 back wall of holster 18
46 curved side wall of holster 18
48 top notch in side wall 46 of holster 18
50 ear piece of stethoscope 16
52 spring clip on back wall 44 of holster 18

### DETAILED DESCRIPTION

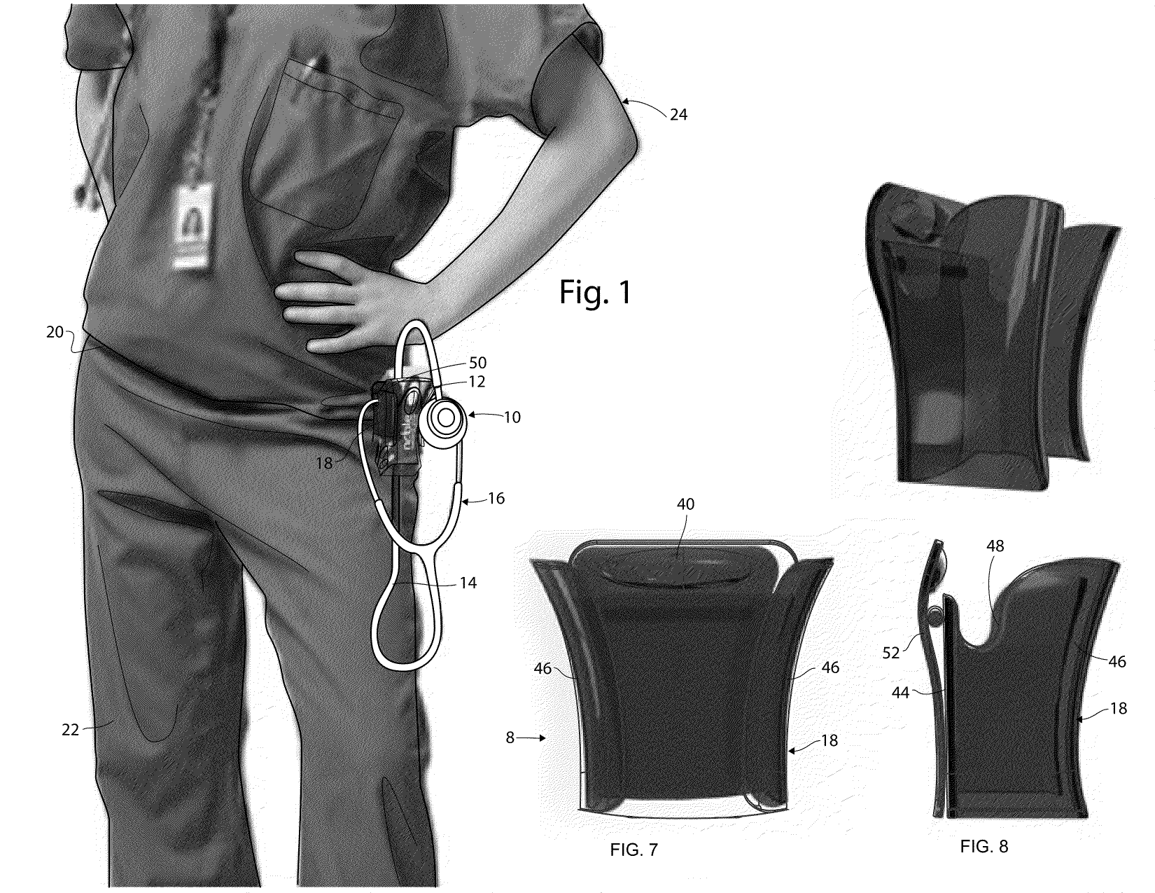

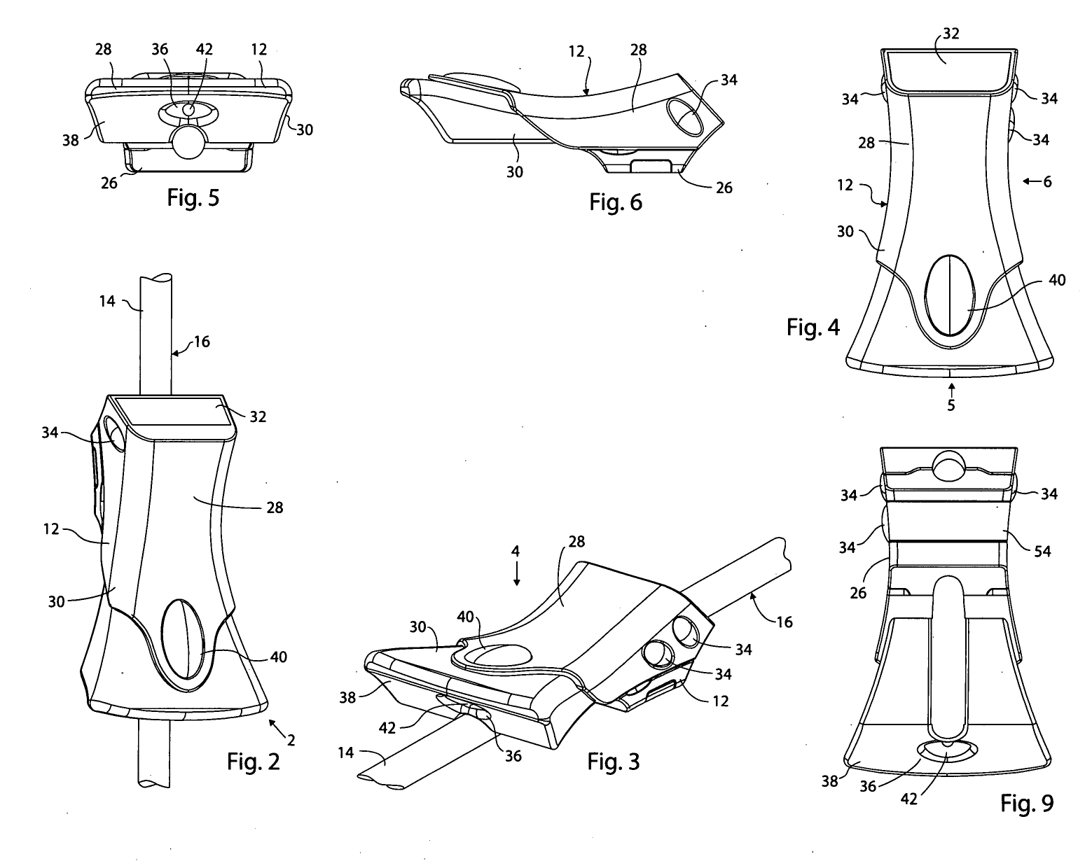

Referring now to the figures, in which like numerals indicate like parts, and particularly to FIGS. 1 through 8, which are a front perspective view of an embodiment of the present invention holding a stethoscope to a waist band of a pair of pants; a front perspective view of the timing device attached to the tube of the stethoscope; a bottom perspective view taken in the direction of arrow 3 in FIG. 2; a front view taken in the direction of arrow 4 in FIG. 3 of the timing device per se; a top view taken in the direction of arrow 5 in FIG. 4; a side view taken in the direction of arrow 6 in FIG. 4; a front view of the holster per se; and a side view taken in the direction of arrow 8 in FIG. 7, FIG. 8 is a side view taken in the direction of arrow 8 in FIG. 7. and FIG. 9 is a back view; and as such, will be discussed with reference thereto.

According to an embodiment, the present invention pertains to a stethoscope timer attachment and clip holder 10 therefore comprising a timing device 12 affixed to a tube 14 of a stethoscope 16. A holster 18 receives the timing device 12 in an inverted position. The holster 18 is connected to a waist band 20 of a pair of pants 22 of a health care professional 24 to carry the stethoscope 16 thereon.

The timing device 12 comprises a snap clamp 26 to removably affix the timing device 12 to the tube 14 of the stethoscope 16. The timing device 12 further comprises a battery operated countdown timer unit 28 therein. The timing device 12 also comprises a housing 30. A digital display screen 32 is in the housing 30. At least one timer button 34 is on the housing 30 to operate the digital display screen 32. An exam light 36 is on the bottom 38 of the housing 30. A button switch 40 is on the housing 30 to turn the exam light 36 on and off. The exam light 36 is a light emitting diode 42.

The holster 18 comprises a back wall 44 and a pair of curved side walls 46 that extend from the back wall 44. Each side wall 46 has a top notch 48 to receive and retain one ear piece 50 of the stethoscope 16 therein. A spring clip 52 is on the back wall 44 of the holster 18 to removably clamp the holster 18 onto the waist band 20 of the pair of pants 22.

It is apparent in view of the teachings herein that embodiments of the invention are particularly useful for attaching a timer to a stethoscope and retaining the timer and the stethoscope by a holster to a waistband of a pair of pants.

It will be understood that each of the elements described above, or two or more together, may also find a useful application in other types of constructions differing from the types described above.

While the invention has been illustrated and described as embodiments of a stethoscope timer attachment and clip holder therefore, accordingly it is not limited to the details shown, since it will be understood that various omissions, modifications, substitutions and changes in the forms and details the device illustrated and its operation can be made by those skilled in the art without departing in any way from the sprint of the present invention. The teachings of all of the references cited herein are incorporated by reference to the extent not inconsistent with the teachings herein.

1. A stethoscope timer attachment and clip holder therefore comprising:
a) a timing device affixed to a tube of a stethoscope; and
b) a holster to receive said timing device in an inverted position, wherein said holster is connected to a waist band of a pair of pants of a health care professional to carry the stethoscope thereon.

2. The attachment as recited in claim 1, wherein said timing device comprises a snap clamp to removably affix said timing device to the tube of the stethoscope.

3. The attachment as recited in claim 1, wherein said timing device further comprises a battery operated countdown timer unit therein.

4. The attachment as recited in claim 1, wherein said timing device further comprises
a) a housing;
b) a digital display screen in said housing; and
c) at least one timer button on said housing to operate said digital display screen.

5. The attachment as recited in claim 4, wherein said timing device further comprises:
a) an exam light on bottom of said housing; and
b) a button switch on said housing to turn said exam light on and off.

6. The attachment as recited in claim 5, wherein said exam light is a light emitting diode.

7. The attachment as recited in claim 1, wherein said holster comprises:
a) a back wall; and
b) a pair of curved side walls extending from said back wall, wherein each said side wall having a top notch will receive and retain one ear piece of the stethoscope therein.

8. The attachment as recited in claim 7, further comprises a spring clip on said back wall of said holster to removably clamp said holster onto the waist band of the pair of pants.

9. A stethoscope carrying system comprising an attachment device affixed to a tube of a stethoscope, said attachment device comprising a housing;
a holster comprising a body that comprises an outer shell that defines a cavity, the body having a top end with an opening sized to receive said attachment device for placement of said attachment device in said cavity; and
a clip associated with said outer shell for engaging said holster to a belt or waist of a user.

10. The stethoscope carrying system of claim 9, wherein said housing comprises a three-dimensional configuration and said body comprises a front wall, back wall, and opposing side walls forming said outer shell and defining said cavity to be substantially similar in shape with said three-dimensional configuration so as to create a frictional fit between said holster and said attachment device.

11. The stethoscope carrying system of claim 10, wherein said side walls each comprise a notch for receiving and supporting an ear piece of a stethoscope.
